(19) United States
(12) Patent Application Publication
(10) Pub. No.: US 2025/0059149 A1
(43) Pub. Date: Feb. 20, 2025

(54) MICROREACTOR AND METHOD FOR CONTINUOUS FLOW SYNTHESIS OF 2,5-FURANDICARBOXYLIC ACID

(71) Applicant: JIANGSU UNIVERSITY OF SCIENCE AND TECHNOLOGY, Jiangsu (CN)

(72) Inventors: Chao YU, Jiangsu (CN); Jialun HE, Jiangsu (CN); Xiao CHEN, Jiangsu (CN); Yingguo LI, Jiangsu (CN); Danfeng JIANG, Jiangsu (CN)

(73) Assignee: JIANGSU UNIVERSITY OF SCIENCE AND TECHNOLOGY, Jiangsu (CN)

(21) Appl. No.: 18/936,914

(22) Filed: Nov. 4, 2024

Related U.S. Application Data
(63) Continuation of application No. PCT/CN2024/078933, filed on Feb. 28, 2024.

(30) Foreign Application Priority Data
Jul. 5, 2023 (CN) 202310821075.5

(51) Int. Cl. C07D 307/68; B01J 19/00

(52) U.S. Cl. CPC C07D 307/68; B01J 19/004

(57) ABSTRACT

Disclosed is a microreactor for continuous flow synthesis of 2,5-furandicarboxylic acid, the microreactor includes a first container, a collection device, an injection pump, a first flow pump, a second flow pump, a first microreactor substrate, ion exchange resin adsorption pipes, activated carbon adsorption pipes, a second microreactor substrate, an oxygen source, a first T-connector, a second T-connector, and a third T-connector; where the first container is connected to the first T-connector via the first flow pump, the injection pump is connected to the first T-connector, the third T-connector is connected to the first T-connector and the first microreactor substrate, respectively, the first microreactor substrate is connected to the ion exchange resin adsorption pipes, the activated carbon adsorption pipes, the second T-connector, and the second microreactor substrate in sequence, and the oxygen source is connected to the second T-connector. Further disclosed is a method for continuous flow synthesis of 2,5-furandicarboxylic acid.

### CROSS-REFERENCE TO RELATED APPLICATION

This application is a continuation of international application of PCT application serial no. PCT/CN2024/078933, filed on Feb. 28, 2024, which claims the priority benefit of China application no. 202310821075.5, filed on Jul. 5, 2023. The entirety of each of the above-mentioned patent applications is hereby incorporated by reference herein and made a part of this specification.

### BACKGROUND

### Technical Field

The present disclosure belongs to the field of preparing 2,5-furandicarboxylic acid, and particularly relates to a microreactor and a method for continuous flow synthesis of 2,5-furandicarboxylic acid.

### Description of Related Art

Nowadays, with the rapid development of global industrialization, the existing fossil fuel, as non-renewable resources, is not only becoming increasingly scarce, but also causes serious environmental issues, forcing relevant practitioners to have to seek for new environmentally friendly alternative materials. Biomass resources, with advantages of abundant reserves, low price, green and environmental protection, and strong renewability, become ideal substitutes for the fossil fuel. 5-hydroxymethylfurfural (HMF) is commonly considered to be an intermediate that link the biomass resources to the petrochemical industry. It can usually be derived from C6 sugars (fructose or glucose) and then synthesized into 2,5-furandicarboxylic acid (FDCA) using an oxidation agent, a metal catalyst, or a biological enzyme.

However, preparing FDCA from the fructose in an efficiently and cost-effective manner present huge challenges, facing various difficulties. Specifically, incomplete oxidation of HMF in the process of synthesizing FDCA will lead to low-quality polymers, undesirable condensation at a moderate temperature makes HMF unstable, therefore, an oxidation process of the HMF can only be performed at a low substrate concentration; and furthermore, multi-step reactions are accompanied by the consumption of energy, solvents, and time, which will increase the production costs. The Chinese Patent (Application No. CN202210877671.0) discloses a method for preparing 2,5-furandicarboxylic acid from fructose without metal catalysis in one pot, but it still requires an alkaline environment and a maximum reaction time of up to 52 hours.

In the prior art, one-step synthesis of 2,5-furandicarboxylic acid from the fructose requires high temperature, high pressure, strong acid, strong alkali, and heavy metal catalyst, and along with a reaction duration of 20-40 hours, and will generate a large amount of by-products, causing environmental pollution and making mass production difficult and hazardous. In addition, step-by-step synthesis of 2,5-furandicarboxylic acid from the fructose will generate a large amount of by-products, resulting in a decrease in yield.

### SUMMARY

Objectives: In order to overcome the deficiencies existing in the prior art, one objective of the present disclosure is to provide a safe, efficient, and environmentally friendly microreactor for continuous flow synthesis of 2,5-furandicarboxylic acid; and the other objective of the present disclosure is to provide a method for continuous flow synthesis of 2,5-furandicarboxylic acid capable of realizing mass production and lowering a reaction temperature.

### Technical Solution

the microreactor for continuous flow synthesis of 2,5-furandicarboxylic acid provided in the present disclosure includes a first container, a collection device, an injection pump, a first flow pump, a second flow pump, a first microreactor substrate, ion exchange resin adsorption pipes, activated carbon adsorption pipes, a second microreactor substrate, an oxygen source, a first T-connector, a second T-connector, and a third T-connector; the first container is connected to the first T-connector via the first flow pump, the injection pump is connected to the first T-connector, the third T-connector is connected to the first T-connector and the first microreactor substrate, respectively, the first microreactor substrate is connected to the ion exchange resin adsorption pipes, the activated carbon adsorption pipes, the second T-connector, and the second microreactor substrate in sequence, the oxygen source is connected to the second T-connector, and the collection device is connected to the third T-connector via the second flow pump, allowing for reuse of a solvent; a reaction temperature for the first microreactor substrate falls within 70-210° C., and a reaction temperature for the second microreactor substrate falls within 40-230° C.; and baffles are provided in S-shaped flow channels of the first microreactor substrate and the second microreactor substrate, such that turbulence can be formed at bends of the S-shaped flow channels, and the baffles.

Further, the first microreactor substrate and the second microreactor substrate are made from metal, plastic or carbon material. The plastic is preferably polytetrafluoroethylene, polyvinylidene fluoride or polyimide, which is heat-resistant and corrosion-resistant; the metal is preferably hastelloy or stainless steel; and the carbon material is preferably silicon carbide. The S-shaped flow channels are S-shaped circular pipelines, with a length of 100-400 mm, an inner diameter of 0.8-1.0 mm, and a spacing smaller than the inner diameter. A flow rate of the reaction solution in the S-shaped channel is 0.01-20 mL/min.

Further, the first container is configured to store a fructose solution with a mass ratio of 5%-15%. An inner diameter of a pipe from which the fructose solution flows out is 0.8-50 mm.

Further, the injection pump contains an HCl solution. The first container and the collection device are both conical flasks.

Further, the ion exchange resin adsorption pipes and the activated carbon adsorption pipes are both glass TVOC tubes, with an inner diameter of 6-600 mm, a length of 150-15000 mm, and a filler volume of 200-60000 mg, respectively. The ion exchange resin adsorption pipes are configured to remove Cl⁻ in the solution, and the activated carbon adsorption pipes are configured to remove humin in the solution.

Further, the oxygen source is connected to a second T-connector via a gas mass flow meter, and the gas mass flow meter has a flow rate of 90-100 mL/min.

A method for continuous flow synthesis of 2,5-furandicarboxylic acid provided in the present disclosure includes the following steps:
step 1: a reaction solution is transferred to the first container, and temperatures of the first microreactor substrate and the second microreactor substrate are adjusted to be 70-210° C. and 40-230° C., respectively; and
step 2: the first flow pump and the injection pump are turned on simultaneously, flow rates of a fructose solution and an HCl solution are adjusted, respectively, such that the fructose solution and the HCl solution form uniform laminar flow in pipelines, the fructose solution and the HCl solution are driven to enter into the third T-connector, mixed in the third T-connector and then directed to flow towards the first microreactor substrate, synthesis of 5-hydroxymethylfurfural is completed in the first microreactor substrate, afterwards, the solution containing 5-hydroxymethylfurfural enters the ion exchange resin adsorption pipes to have Cl-removed, and then enters the activated carbon adsorption pipes, the oxygen source is turned on, a gas flow rate is monitored using the gas mass flow meter; and after the adsorption is completed, and the solution enters the second microreactor substrate via the second T-connector for oxidation; and upon completion of the reaction, a solid phase product in the collection device is cooled and collected, the solvent is returned to the third T-connector through the second flow pump for reuse, achieving a yield of over 90%.

Further, residence time of materials in the first microreactor substrate and the second microreactor substrate is less than or equal to 60 min. The oxygen source is not limited to input from an oxygen cylinder, but also includes hydrogen production from hydrogen peroxide, methanol decomposition to hydrogen, water electrolysis for hydrogen production, methane steam reforming for hydrogen production, and ammonia decomposition for hydrogen production.

The present disclosure aims to provide a method for synthesizing 2,5-furandicarboxylic acid from fructose using continuous flow. In conventional methods, fructose generates 5-hydroxymethylfurfural in an acidic environment; however, the synthesis of 2,5-furandicarboxylic acid from 5-hydroxymethylfurfural requires an alkaline environment. The difficulty of the method lies in how to synthesize the target product in both an acidic and alkaline environment in a continuous process. The present disclosure adopts a catalyst to avoid the use of strong alkali in the synthesis process of converting 5-hydroxymethylfurfural to 2,5-furandicarboxylic acid, and removes the residual byproducts and ions of fructose synthesis of 5-hydroxymethylfurfural through activated carbon adsorption pipes and ion exchange resin adsorption pipes, thereby achieving efficient synthesis of fructose to 2,5-furandicarboxylic acid from fructose.

Principle for preparation: the present disclosure uses fructose as raw material and adopts microreactor technology to synthesize 2,5-furandicarboxylic acid. Microfluidic technology not only has the characteristics of miniaturization and integration, but also has the advantages of small size, consumption of few material and energy, fast reaction speed, and good continuity. By using the microreactor, the reaction conditions can be precisely controlled and different organic synthesis conditions can be adjusted, such that different batches of products can be produced continuously. In addition, reactions at a microscale can also avoid scale-up effects in chemical production and significantly improve the mass and heat transfer efficiency. The synthesis method of the present disclosure optimizes the method of organic synthesis by regulating the reaction conditions through microscale means and enhancing the mixing effects of the fluid with baffles, thereby reducing the reaction conditions, shortening the reaction time, and increasing the yield and production rate of 2,5-furandicarboxylic acid.

### Beneficial Effects

compared with the prior art, the present disclosure has the remarkable features as follows:
1. The reaction is controllable, continuous, suitable in reaction temperature, and fast in synthesis time, and achieves fast synthesis and batch production, and reaction conditions such as reaction temperature and reaction time can be accurately controlled, making the entire synthesis process occurs in an alkali-free environment, and the production of 2,5-furandicarboxylic acid more environmentally friendly.
2. The reaction temperature can be lowered, and the one-pot synthesis process avoids waste of raw material and can be performed in a sealed environment; and continuous flow can promote the reaction, such that the reactions that were previously high-risk and difficult can be carried out in an efficient and safe manner.
3. Pipelines and materials of the first and second microreactor substrates can be customized according to the synthesis requirements, with low cost and small pipeline dimensions, which improve mass and heat transfer efficiency, as well as reaction efficiency.
4. The method features high yield, high selectivity, and high energy utilization rate, and the existing methods require 20-40 h for synthesis, but the present disclosure shortens the reaction time to 60-180 min.
s

### BRIEF DESCRIPTION OF THE DRAWINGS

FIG. 1 is a structural schematic diagram of the present disclosure.

FIG. 2 is a structural schematic diagram of a first microreactor substrate according to the present disclosure.

FIG. 3 shows yields of 2,5-furandicarboxylic acid (FDCA) and 2-fluoro-4-chlorobenzoic acid when GVL:H₂O is 80:20 in the present disclosure.

FIG. 4 shows yields of 2,5-furandicarboxylic acid and 2-fluoro-4-chlorobenzoic acid when GVL:H₂O is 50:50 in the present disclosure.

FIG. 5 shows conversion rates of fructose in an acidic environment created by HCl and 2,5-furandicarboxylic acid, respectively.

### DESCRIPTION OF THE EMBODIMENTS

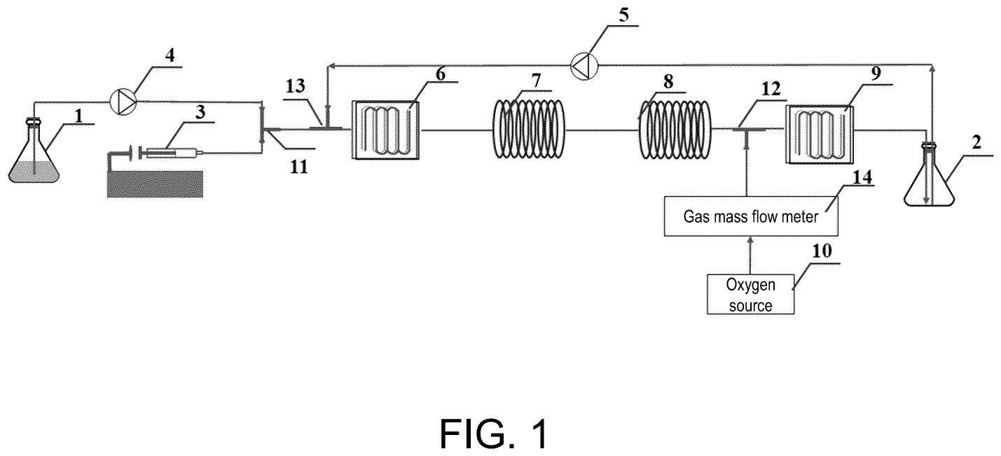

As shown in FIG. 1, a microreactor for continuous flow synthesis of 2,5-furandicarboxylic acid is provided, where a first container 1 is configured to store a reaction solution, that is, a fructose solution in a mass ratio of 5%-15%, and a collection device 2 is configured to collect a product, that is, a 2,5-furandicarboxylic acid crystal and a 2,5-furandicarboxylic acid solution. The first container 1 is connected to a first T-connector 11 via a first flow pump 4, an injection pump 3 is connected to the first T-connector 11, a third T-connector 13 is connected to the first T-connector 11 and a first microreactor substrate 6, respectively, the first microreactor substrate 6 is connected to ion exchange resin adsorption pipes 7, activated carbon adsorption pipes 8, a second T-connector 12, and a second microreactor substrate 9 in sequence, Cl⁻ in the 5-hydroxymethylfurfural enter solution is removed in the ion exchange resin adsorption pipes 7, and humin is removed in the activated carbon adsorption pipes 8. An oxygen source 10 is connected to a second T-connector 12 via a gas mass flow meter, and the gas mass flow meter has a flow rate of 90-100 mL/min. The collection device 2 is connected to the third T-connector 13 via a second flow pump 5, allowing for reuse of a solvent. A reaction temperature for the first microreactor substrate 6 falls within 70-210° C., while a reaction temperature for the second microreactor substrate 9 falls within 40-230° C. Baffles are provided in S-shaped flow channels of the first microreactor substrate 6 and the second microreactor substrate 9. An inner diameter of a pipe from which the fructose solution flows out is 0.8-50 mm.

The first container 1 and the collection device 2 are both common laboratory conical flasks with specifications of 100-200 mL. The injection pump 3 contains an HCl solution, the first flow pump 4 contains a first reaction solution, and the first reaction solution and the HCl solution are preliminarily mixed at the first T-connector 11. The second flow pump 5 contains a 2,5-furandicarboxylic acid solution, which is mixed with the first reaction solution at the third T-connector 13.

The ion exchange resin adsorption pipes 7 and the activated carbon adsorption pipes 8 are both glass TVOC tubes, with an inner diameter of 6-600 mm, a length of 150-15000 mm, and a filler volume of 200-60000 mg, respectively. The microreactor adopts a structure that 1-5 activated carbon adsorption pipes 8 (or the ion exchange resin adsorption pipes 7) are arranged in each row, with a total of 2-4 rows, the pipes are connected together using three-way or five-way valves, and adsorption time for each of the adsorption tubes is 2-600 h. After the adsorption time of adsorption tubes is reached, one of the valves is turned to direct the reaction solution to other valves, and the original adsorption tubes are washed and replaced. The multi-row structure facilitates the replacement of the adsorption tubes.

5-hydroxymethylfurfural is synthesized in the first microreactor substrate 6, a yield thereof can be greater than 90%, and a conversion rate can be greater than 99%, excluding the 5-hydroxymethylfurfural. The 5-hydroxymethylfurfural is oxidized in the second microreactor substrate 9 to obtain the 2,5-furandicarboxylic acid, with a final yield greater than 90% and a conversion rate greater than 99%.

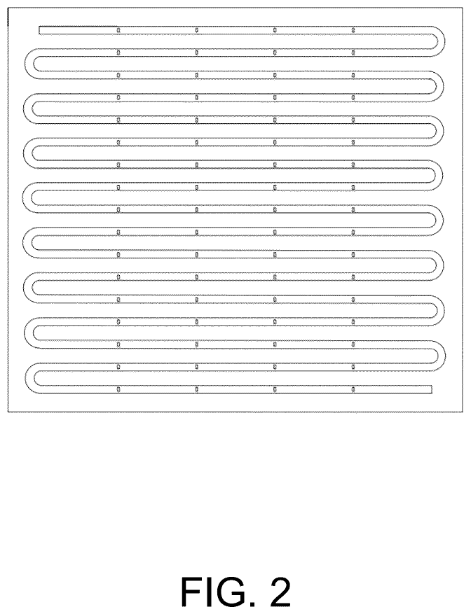

As shown in FIG. 2, structures of the first microreactor substrate 6 and the second microreactor substrate 9 are exactly the same. Taking the first microreactor substrate 6 as an example, the first microreactor substrate 6 contains the S-shaped flow channel with 11-22 channels, and 68-136 micro baffles, and turbulence generated at 22-44 corner points and the 68-136 micro baffles can stir the reaction solution, accelerating a reaction process. The first microreactor substrate 6 and the second microreactor substrate 9 are made from metal, plastic or carbon material. The plastic is preferably polytetrafluoroethylene, polyvinylidene fluoride or polyimide, which is heat-resistant and corrosion-resistant; the metal is preferably Hastelloy or stainless steel; and the carbon material is preferably silicon carbide. Flow rates of the injection pump 3, the first flow pump 4, and the second flow pump 5 are adjusted, and residence time is controlled to 60 min according to Formula 1 and Formula 2. The S-shaped flow channels are S-shaped circular pipelines, with a length of 100-400 mm, an inner diameter of 0.8-1.0 mm, and a spacing smaller than the inner diameter. A flow rate of the reaction solution in the S-shaped channel is 0.01-20 mL/min. Each of the baffles has a length of 0.4-20 mm, and a width of 0.2-10 mm. A microchannel has a high specific surface area and a very small microchannel characteristic dimension, providing excellent heat and mass transfer characteristics, which are conducive to controlling the reaction process on a microscopic scale, improving selectivity and yield of the reaction.

$$\overline{t} = {\int }_{0}^{∞} tE ( t ) dt \qquad ( Formula 1 )$$

$${σ}^{2} = {\int }_{0}^{∞} {t}^{2} E ( t ) dt - {t}^{2} \qquad ( Formula 2 )$$

where t̅ is average residence time, t is residence time, E(t) is a residence time distribution density function, and σ is a variance of residence time distribution.

### Example 1

A method for continuous flow synthesis of 2,5-furandicarboxylic acid, including the following steps:
S1. a reaction solution was prepared: 50 ml of γ-valerolactone (GVL) and 50 ml of Milli-Q water were respectively weighed and taken as a solvent, 0.88 g of fructose was weighed and added into the solvent to prepare a fructose solution in a mass ratio of 15% as the reaction solution;
S2. a catalyst was synthesized: carbon was thoroughly washed with Milli-Q water, and then dried in a vacuum oven (323 K, 500 mbar). Hexachloroplatinate (IV) dihydrogen hexahydrate was used as a Pt precursor, without further treatment. H₂PtCl₆·6H₂O was loaded onto the carbon. 0.135 g of H₂PtCl₆·6H₂O was weighed for each 1 g of carbon, and dissolved into the 1.7 of Milli-Q water to reach a wet point of the carbon (1.7 ml/g carbon). The precursor solution dissolved was added to a carbon carrier, and the carbon carrier impregnated was dried at 383 K. The catalyst was treated with flowing H₂ (100 ml/min) for 3 h, and then passivated with 1% O₂ at room temperature and argon conditions;
S3. a first reaction solution was prepared and transferred to the first container 1, a heating device was turned on, and temperatures of the first microreactor substrate 6 and the second microreactor substrate 9 were adjusted to be 110° C. and 180° C., respectively; and
S4. the first flow pump 4 and the injection pump 3 were turned on, such that the first reaction solution and an HCl solution formed a required flow pattern in pipelines. The fructose solution and the HCl solution were driven to enter into the third T-connector 13, mixed in the third T-connector 13 and then directed to flow towards the first microreactor substrate 6, and synthesis of 5-hydroxymethylfurfural was completed within 30 min in the first microreactor substrate 6 under combined effects of the baffles and an original flow rate; and the first microreactor substrate 6 contained the S-shaped flow channel with 11 channels and 68 micro baffles, and turbulence generated at 22 corner points and the 68 micro baffles could stir the reaction solution, accelerating a reaction process. Afterwards, the solution containing 5-hydroxymethylfurfural entered the ion exchange resin adsorption pipes 7 to have Cl-removed, and then entered the activated carbon adsorption pipes 8, the oxygen source 10 was turned on, oxygen was directly provided by a gas cylinder to absorb humin in the solution and increase oxygen content in subsequent pipelines, and a gas flow rate was monitored using the gas mass flow meter; and after the adsorption was completed, the reaction solution entered the second microreactor substrate 9 made of hastelloy via the second T-connector 12 for oxidation. 2,5-furandicarboxylic acid was synthesized within 1 h in a pipeline of the second microreactor substrate 9, and finally entered the collection device 2; and after entering the collection device 2, the solution was cooled down to room temperature, a crystal of 2,5-furandicarboxylic acid with a purity>99% was then precipitated from the solution, the remaining solution contained 5% 2,5-furandicarboxylic acid, and returned to the third T-connector 13 via the second flow pump 5, and then entered the first microreactor substrate 6 for reuse.

### Example 2

A method for continuous flow synthesis of 2,5-furandicarboxylic acid, including the following steps:
S1. a reaction solution was prepared: 50 ml of γ-valerolactone (GVL) and 50 ml of Milli-Q water were respectively weighed and taken as a solvent, 0.29 g of fructose was weighed and added into the solvent to prepare a fructose solution in a mass ratio of 5% as the reaction solution;
S2. a catalyst was synthesized: carbon was thoroughly washed with Milli-Q water, and then dried in a vacuum oven (323 K, 500 mbar). Hexachloroplatinate (IV) dihydrogen hexahydrate was used as a Pt precursor, without further treatment. H₂PtCl₆·6H₂O was loaded onto the carbon. 0.135 g of H₂PtCl₆·6H₂O was weighed for each 1 g of carbon, and dissolved into the 1.7 of Milli-Q water to reach a wet point of the carbon (1.7 ml/g carbon). The precursor solution dissolved was added to a carbon carrier, and the carbon carrier impregnated was dried at 383 K. The catalyst was treated with flowing H₂ (100 ml/min) for 3 h, and then passivated with 1% O₂ at room temperature and argon conditions;
S3. a first reaction solution was prepared and transferred to the first container 1, a heating device was turned on, and temperatures of the first microreactor substrate 6 and the second microreactor substrate 9 were adjusted to be 210° C. and 230° C., respectively; and
S4. the first flow pump 4 and the injection pump 3 were turned on, such that the first reaction solution and an HCl solution formed a required flow pattern in pipelines. The fructose solution and the HCl solution were driven to enter into the third T-connector 13, mixed in the third T-connector 13 and then directed to flow towards the first microreactor substrate 6, and synthesis of 5-hydroxymethylfurfural was completed within 25 min in the first microreactor substrate 6 under combined effects of the baffles and an original flow rate; and the first microreactor substrate 6 contained the S-shaped flow channel with 22 channels and 136 micro baffles, and turbulence generated at 44 corner points and the 136 micro baffles could stir the reaction solution, accelerating a reaction process. Afterwards, the solution containing 5-hydroxymethylfurfural entered the ion exchange resin adsorption pipes 7 to have Cl-removed, and then entered the activated carbon adsorption pipes 8, the oxygen source 10 was turned on, oxygen was directly provided by a gas cylinder to absorb humin in the solution and increase oxygen content in subsequent pipelines, and a gas flow rate was monitored using the gas mass flow meter; and after the adsorption was completed, the solution entered the second microreactor substrate 9 made of hastelloy via the second T-connector 12 for oxidation. 2,5-furandicarboxylic acid was synthesized within 1.5 h in a pipeline of the second microreactor substrate 9, and finally entered the collection device 2; and after entering the collection device 2, the solution was cooled down to room temperature, a crystal of 2,5-furandicarboxylic acid with a purity>99% was then precipitated from the solution, the remaining solution contained 5% 2,5-furandicarboxylic acid, and returned to the third T-connector 13 via the second flow pump 5, and then entered the first microreactor substrate 6 for reuse.

### Example 3

A method for continuous flow synthesis of 2,5-furandicarboxylic acid, including the following steps:
S1. a reaction solution was prepared: 50 ml of γ-valerolactone (GVL) and 50 ml of Milli-Q water were respectively weighed and taken as a solvent, 0.88 g of fructose was weighed and added into the solvent to prepare a fructose solution in a mass ratio of 15% as the reaction solution;
S2. a catalyst was synthesized: carbon was thoroughly washed with Milli-Q water, and then dried in a vacuum oven (323 K, 500 mbar). Hexachloroplatinate (IV) dihydrogen hexahydrate was used as a Pt precursor, without further treatment. H₂PtCl₆·6H₂O was loaded onto the carbon. 0.135 g of H₂PtCl₆·6H₂O was weighed for each 1 g of carbon, and dissolved into the 1.7 of Milli-Q water to reach a wet point of the carbon (1.7 ml/g carbon). The precursor solution dissolved was added to a carbon carrier, and the carbon carrier impregnated was dried at 383 K. The catalyst was treated with flowing H₂ (100 ml/min) for 3 h, and then passivated with 1% O₂ at room temperature and argon conditions;
S3. a first reaction solution was prepared and transferred to the first container 1, a heating device was turned on, and temperatures of the first microreactor substrate 6 and the second microreactor substrate 9 were adjusted to be 130° C. and 160° C., respectively; and
S4. the first flow pump 4 and the injection pump 3 were turned on, such that the first reaction solution and an HCl solution formed a required flow pattern in pipelines. The fructose solution and the HCl solution were driven to enter into the third T-connector 13, mixed in the third T-connector 13 and then directed to flow towards the first microreactor substrate 6, and synthesis of 5-hydroxymethylfurfural was completed within 60 min in the first microreactor substrate 6 under combined effects of the baffles and an original flow rate; and the first microreactor substrate 6 contained the S-shaped flow channel with 22 channels and 136 micro baffles, and turbulence generated at 44 corner points and the 136 micro baffles could stir the reaction solution, accelerating a reaction process. Afterwards, the solution containing 5-hydroxymethylfurfural entered the ion exchange resin adsorption pipes 7 to have Cl-removed, and then entered the activated carbon adsorption pipes 8, the oxygen source 10 was turned on, oxygen was directly provided by a gas cylinder to absorb humin in the solution and increase oxygen content in subsequent pipelines, and a gas flow rate was monitored using the gas mass flow meter; and after the adsorption was completed, the solution entered the second microreactor substrate 9 made of hastelloy via the second T-connector 12 for oxidation. 2,5-furandicarboxylic acid was synthesized within 2 h in a pipeline of the second microreactor substrate 9, and finally entered the collection device 2; and after entering the collection device 2, the solution was cooled down to room temperature, a crystal of 2,5-furandicarboxylic acid with a purity>99% was then precipitated from the solution, the remaining solution contained 5% 2,5-furandicarboxylic acid, and returned to the third T-connector 13 via the second flow pump 5, and then entered the first microreactor substrate 6 for reuse.

### Example 4

A method for continuous flow synthesis of 2,5-furandicarboxylic acid, including the following steps:
S1. a reaction solution was prepared: 50 ml of γ-valerolactone (GVL) and 50 ml of Milli-Q water were respectively weighed and taken as a solvent, 0.88 g of fructose was weighed and added into the solvent to prepare a fructose solution in a mass ratio of 15% as the reaction solution;
S2. a catalyst was synthesized carbon was thoroughly washed with Milli-Q water, and then dried in a vacuum oven (323 K, 500 mbar). Hexachloroplatinate (IV) dihydrogen hexahydrate was used as a Pt precursor, without further treatment. H₂PtCl₆·6H₂O was loaded onto the carbon. 0.135 g of H₂PtCl₆·6H₂O was weighed for each 1 g of carbon, and dissolved into the 1.7 of Milli-Q water to reach a wet point of the carbon (1.7 ml/g carbon). The precursor solution dissolved was added to a carbon carrier, and the carbon carrier impregnated was dried at 383 K. The catalyst was treated with flowing H₂ (100 ml/min) for 3 h, and then passivated with 1% O₂ at room temperature and argon conditions;
S3. a first reaction solution was prepared and transferred to the first container 1, a heating device was turned on, and temperatures of the first microreactor substrate 6 and the second microreactor substrate 9 were adjusted to be 210° C. and 230° C., respectively; and
S4. the first flow pump 4 and the injection pump 3 were turned on, such that the first reaction solution and an HCl solution formed a required flow pattern in pipelines. The fructose solution and the HCl solution were driven to enter into the third T-connector 13, mixed in the third T-connector 13 and then directed to flow towards the first microreactor substrate 6, and synthesis of 5-hydroxymethylfurfural was completed within 45 min in the first microreactor substrate 6 under combined effects of the baffles and an original flow rate; and the first microreactor substrate 6 contained the S-shaped flow channel with 11 channels and 68 micro baffles, and turbulence generated at 22 corner points and the 68 micro baffles could stir the reaction solution, accelerating a reaction process. Afterwards, the solution containing 5-hydroxymethylfurfural entered the ion exchange resin adsorption pipes 7 to have Cl-removed, and then entered the activated carbon adsorption pipes 8, the oxygen source 10 was turned on, oxygen was directly provided by a gas cylinder to absorb humin in the solution and increase oxygen content in subsequent pipelines, and a gas flow rate was monitored using the gas mass flow meter; and after the adsorption was completed, the solution entered the second microreactor substrate 9 made of hastelloy via the second T-connector 12 for oxidation. 2,5-furandicarboxylic acid was synthesized within 1.5 h in a pipeline of the second microreactor substrate 9, and finally entered the collection device 2; and after entering the collection device 2, the solution was cooled down to room temperature, a crystal of 2,5-furandicarboxylic acid with a purity>99% was then precipitated from the solution, the remaining solution contained 5% 2,5-furandicarboxylic acid, and returned to the third T-connector 13 via the second flow pump 5, and then entered the first microreactor substrate 6 for reuse.

### Example 5

A method for continuous flow synthesis of 2,5-furandicarboxylic acid, including the following steps:
S1. a reaction solution was prepared: 50 ml of γ-valerolactone (GVL) and 50 ml of Milli-Q water were respectively weighed and taken as a solvent, 0.88 g of fructose was weighed and added into the solvent to prepare a fructose solution in a mass ratio of 15% as the reaction solution;
S2. a catalyst was synthesized: carbon was thoroughly washed with Milli-Q water, and then dried in a vacuum oven (323 K, 500 mbar). Hexachloroplatinate (IV) dihydrogen hexahydrate was used as a Pt precursor, without further treatment. H₂PtCl₆·6H₂O was loaded onto the carbon. 0.135 g of H₂PtCl₆·6H₂O was weighed for each 1 g of carbon, and dissolved into the 1.7 of Milli-Q water to reach a wet point of the carbon (1.7 ml/g carbon). The precursor solution dissolved was added to a carbon carrier, and the carbon carrier impregnated was dried at 383 K. The catalyst was treated with flowing H₂ (100 ml/min) for 3 h, and then passivated with 1% O₂ at room temperature and argon conditions;
S3. a first reaction solution was prepared and transferred to the first container 1, a heating device was turned on, and temperatures of the first microreactor substrate 6 and the second microreactor substrate 9 were adjusted to be 70° C. and 40° C., respectively; and
S4. the first flow pump 4 and the injection pump 3 were turned on, such that the first reaction solution and an HCl solution formed a required flow pattern in pipelines. The fructose solution and the HCl solution were driven to enter into the third T-connector 13, mixed in the third T-connector 13 and then directed to flow towards the first microreactor substrate 6, and synthesis of 5-hydroxymethylfurfural was completed within 30 min in the first microreactor substrate 6 under combined effects of the baffles and an original flow rate; and the first microreactor substrate 6 contained the S-shaped flow channel with 11 channels and 68 micro baffles, and turbulence generated at 22 corner points and the 68 micro baffles could stir the reaction solution, accelerating a reaction process. Afterwards, the solution containing 5-hydroxymethylfurfural entered the ion exchange resin adsorption pipes 7 to have Cl-removed, and then entered the activated carbon adsorption pipes 8, the oxygen source 10 was turned on, oxygen was directly provided by a gas cylinder to absorb humin in the solution and increase oxygen content in subsequent pipelines, and a gas flow rate was monitored using the gas mass flow meter; and after the adsorption was completed, the solution entered the second microreactor substrate 9 made of hastelloy via the second T-connector 12 for oxidation. 2,5-furandicarboxylic acid was synthesized within 30 min in a pipeline of the second microreactor substrate 9, and finally entered the collection device 2; and after entering the collection device 2, the solution was cooled down to room temperature, a crystal of 2,5-furandicarboxylic acid with a purity>99% was then precipitated from the solution, the remaining solution contained 5% 2,5-furandicarboxylic acid, and returned to the third T-connector 13 via the second flow pump 5, and then entered the first microreactor substrate 6 for reuse. Among the above examples, the method for synthesis in Example 2 was the best one.

### Comparative Example 1

In this comparative example, all other steps were the same as those in Example 2, except that 80 ml of γ-valerolactone (GVL) and 20 ml of Milli-Q water were used as a solvent.

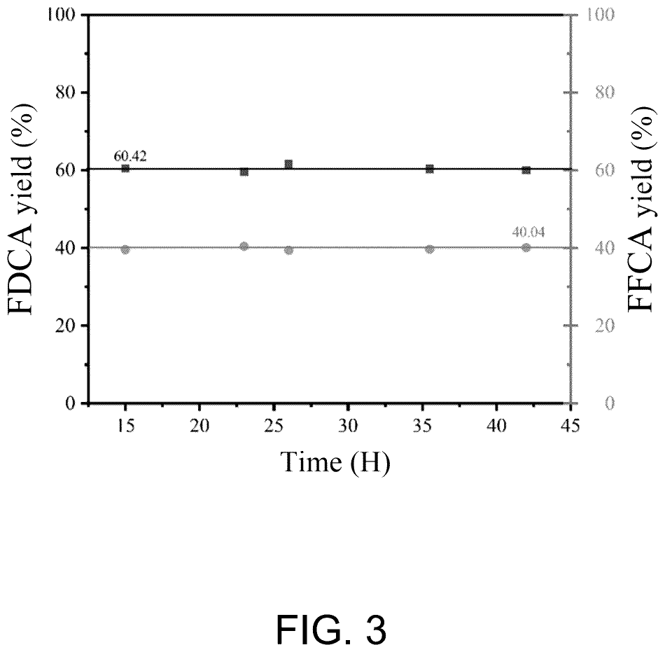

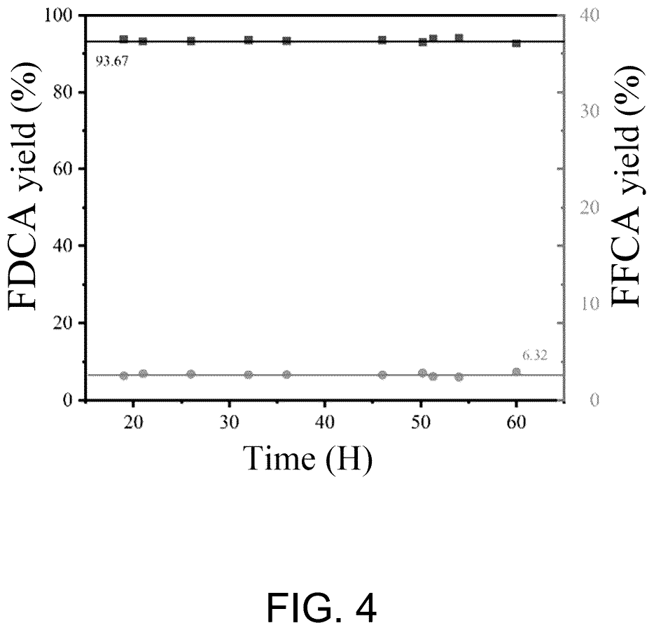

For a sample 1, 80 ml of γ-valerolactone (GVL) and 20 ml of Milli-Q water were used as a solvent, and 0.88 g of fructose was weighed and added to the solvent to prepare a fructose solution in a mass ratio of 15%; and for a sample 2, 50 ml of γ-valerolactone (GVL) and 50 ml of Milli-Q water were respectively weighed and taken as a solvent, and 0.88 g of fructose was weighed and added into the solvent to prepare a fructose solution in a mass ratio of 15% as the reaction solution. Other steps were the same as those in Example 2, samples were taken every one hour to calculate a yield, and the sample 1 was plotted in FIG. 3, and the sample 2 was plotted in FIG. 4. As can be seen from FIGS. 3-4, when 80 ml of γ-valerolactone (GVL) and 20 ml of Milli-Q water were used as the solvent, the yield of furandicarboxylic acid was significantly lower than the yield of 2,5-furandicarboxylic acid when 50 ml of γ-valerolactone (GVL) and 50 ml of Milli-Q water were used as the solvent. Therefore, 50 ml of γ-valerolactone (GVL) and 50 ml of Milli-Q water were used as a solvent for subsequent reactions.

### Comparative Example 2

In this comparative example, all other steps were the same as those in Example 2, except that hydrochloric acid was replaced with 2,5-furandicarboxylic acid.

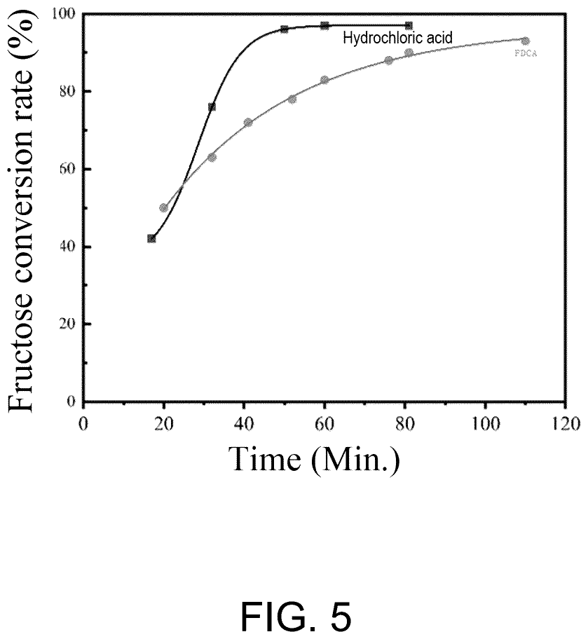

As shown in FIG. 5, hydrochloric acid was used as acid for the sample 1 in the first microreactor plate, while 2,5-furandicarboxylic acid was used as acid for the sample 2 in the first microreactor plate. Other steps were the same as those in Example 2, samples were taken every 10 min to calculate a conversion rate, and the sample 1 was represented by a black line, and the sample 2 was represented by a gray line, as shown in FIG. 5. As can be seen from FIG. 5, a higher fructose conversion rate could be achieved by using 2,5-furandicarboxylic acid, meeting the condition for realizing an overall reuse with 2,5-furandicarboxylic acid.

What is claimed is:

1. A microreactor for continuous flow synthesis of 2,5-furandicarboxylic acid, comprising:
a first container (1), a collection device (2), an injection pump (3), a first flow pump (4), a second flow pump (5), a first microreactor substrate (6), ion exchange resin adsorption pipes (7), activated carbon adsorption pipes (8), a second microreactor substrates (9), an oxygen source (10), a first T-connector (11), a second T-connector (12), and a third T-connector (13);
the first container (1) is connected to the first T-connector (11) via the first flow pump (4), the injection pump (3) is connected to the first T-connector (11), the third T-connector (13) is connected to the first T-connector (11) and the first microreactor substrate (6), respectively, the first microreactor substrate (6) is connected to the ion exchange resin adsorption pipes (7), the activated carbon adsorption pipes (8), the second T-connector (12), and the second microreactor substrates (9) in sequence, the oxygen source (10) is connected to the second T-connector (12), and the collection device (2) is connected to the third T-connector (13) via the second flow pump (5);
a reaction temperature for the first microreactor substrate (6) falls within 70-210° C., and a reaction temperature for the second microreactor substrates (9) falls within 40-230° C.; and
baffles are provided in S-shaped flow channels of the first microreactor substrate (6) and the second microreactor substrates (9).

2. The microreactor for continuous flow synthesis of 2,5-furandicarboxylic acid according to claim 1, wherein the first microreactor substrate (6) and the second microreactor substrate (9) are made from metal, plastic or carbon material.

3. The microreactor for continuous flow synthesis of 2,5-furandicarboxylic acid according to claim 1, wherein the S-shaped flow channels are S-shaped circular pipelines, with a length of 100-400 mm, an inner diameter of 0.8-1.0 mm, and a spacing smaller than the inner diameter.

4. The microreactor for continuous flow synthesis of 2,5-furandicarboxylic acid according to claim 1, wherein the first container (1) is configured to store a fructose solution with a mass ratio of 5%-15%.

5. The microreactor for continuous flow synthesis of 2,5-furandicarboxylic acid according to claim 1, wherein the injection pump (3) contains an HCl solution.

6. The microreactor for continuous flow synthesis of 2,5-furandicarboxylic acid according to claim 1, wherein the first container (1) and the collection device (2) are both conical flasks.

7. The microreactor for continuous flow synthesis of 2,5-furandicarboxylic acid according to claim 1, wherein the ion exchange resin adsorption pipes (7) and the activated carbon adsorption pipes (8) are both glass TVOC tubes, with an inner diameter of 6-600 mm, a length of 150-15000 mm, and a filler volume of 200-60000 mg, respectively.

8. The microreactor for continuous flow synthesis of 2,5-furandicarboxylic acid according to claim 1, wherein the oxygen source (10) is connected to the second T-connector (12) via a gas mass flow meter, and the gas mass flow meter has a flow rate of 90-100 mL/min.

9. A method for continuous flow synthesis of 2,5-furandicarboxylic acid, comprising following steps:
step 1: transferring a reaction solution to a first container (1), and adjusting temperatures of a first microreactor substrate (6) and a second microreactor substrate (9) to be 70-210° C. and 40-230° C., respectively; and
step 2: turning on a first flow pump (4) and an injection pump (3) simultaneously, adjusting flow rates of a fructose solution and an HCl solution, respectively, such that the fructose solution and the HCl solution form uniform laminar flows in pipelines, driving the fructose solution and the HCl solution to enter into a third T-connector (13), mixed in the third T-connector (13) and then directed to flow towards the first microreactor substrate (6), and a synthesis of 5-hydroxymethylfurfural is completed in the first microreactor substrate (6), afterwards, a solution containing 5-hydroxymethylfurfural enters ion exchange resin adsorption pipes (7) to remove Cl⁻, and then enters activated carbon adsorption pipes (8), an oxygen source (10) is turned on, a gas flow rate is monitored using a gas mass flow meter; and after an adsorption is completed, the solution enters the second microreactor substrates (9) via a second T-connector (12) for oxidation;
and upon completion of a reaction, a solid phase product in a collection device (2) is cooled and collected, a solvent is returned to the third T-connector (13) through a second flow pump (5) for reuse.

10. The method for continuous flow synthesis of 2,5-furandicarboxylic acid according to claim 9, wherein a residence time of materials in the first microreactor substrate (6) and the second microreactor substrate (9) is less than or equal to 60 min.
